(19) United States
(12) Patent Application Publication
(10) Pub. No.: US 2026/0226453 A1
(43) Pub. Date: Aug. 6, 2026

(54) Method of SElection with Modified Aptamers (SELMA) Enabling Incorporation of Fluorinated or Methoxylated RNA

(71) Applicant: Brandeis University, Waltham, MA (US)

(72) Inventors: Isaac J. Krauss, Needham Heights, MA (US); Kate B. Fitzpatrick, Cohasset, MA (US); Emily D. Ziperman, Coppell, TX (US)

(73) Assignee: Brandeis University, Waltham, MA (US)

(21) Appl. No.: 19/466,008

(22) Filed: Jan. 30, 2026

Related U.S. Application Data
(60) Provisional application No. 63/752,276, filed on Jan. 31, 2025.

(51) Int. Cl. C12N 15/10; C12N 15/115; C12Q 1/34

(52) U.S. Cl. CPC C12N 15/1048; C12N 15/115; C12Q 1/34; C12N 2310/16; C12N 2310/322; C12N 2310/351; C12N 2310/531; G01N 2333/922

(57) ABSTRACT

Described herein is a method for selecting fully modified RNA aptamers that bind to a target molecule without reverse transcription wherein the method includes providing a first pool of oligonucleotide complexes that each comprise a ds-DNA molecule tethered to a fully modified RNA molecule, wherein the ds-DNA molecule comprises a coding strand and a complementary non-coding strand which encode the RNA molecule. A fully modified RNA molecule includes 2′-fluoro-nucleotides, 2′-methoxy-nucleotides, or a combination of 2′-fluoro-nucleotides and 2′-methoxy-nucleotides, and no un-modified nucleotides. Also included are methods of making a selection library.

### CROSS-REFERENCE TO RELATED APPLICATIONS

This application claims priority to U.S. Provisional Application 63/752,276 filed on Jan. 31, 2025, which is incorporated herein by reference in its entirety.

### STATEMENT REGARDING FEDERALLY SPONSORED RESEARCH & DEVELOPMENT

This invention was made with government support under GM139798, GM151492 awarded by the National Institutes of Health, and 2139752 awarded by the National Science Foundation. The government has certain rights in the invention.

### SEQUENCE LISTING

The Instant Application contains a Sequence Listing which has been submitted electronically in XML format and is hereby incorporated by reference in its entirety. Said XML copy, created on Jan. 27, 2026, is named “SEQ_LIST--107648066.xml” and is 13,658 bytes in size. The Sequence Listing does not go beyond the disclosure in the application as filed.

### FIELD OF THE DISCLOSURE

The present disclosure is related to a method of aptamer selection that enables 2′-fluoro and/or 2′-methoxy ribose substitutions at all positions in the aptamer, providing selection of nuclease-resistant aptamers.

### BACKGROUND

Aptamers are genetically encodable polymers, usually oligonucleotides, that have been selected in vitro from a random library to bind to a protein or molecule of interest. Oligonucleotide aptamers are typically 20-100 bases in length and can achieve high binding affinities for their targets due to their specific folding, shape complementarity, and positioning of functional groups. Aptamers possess several advantages compared with antibodies, including straightforward synthesis and quality control, low immunogenicity, and small size. Aptamers are typically discovered using SELEX (Systematic Evolution of Ligands by EXponential Enrichment), in which libraries of random DNA or RNA sequences flanked by constant primer regions are incubated with a target of interest and the bound sequences are amplified by PCR. Two aptamer therapeutics have been approved for clinical use. Pegaptanib targets vascular endothelial growth factor (VEGF) for the treatment of age-related macular degeneration. In 2023, avacincaptad pegol, an aptamer targeting complement protein C5, was approved for the treatment of geographic atrophy (GA) secondary to age-related macular degeneration.

A barrier to the development of oligonucleotide aptamer therapeutics has been the instability of naked RNA or DNA to nucleases in serum. To address this, aptamers have been extensively stabilized with modified nucleotides (XNA), such as fluoro-(2′-F-) or methoxy (2′-OMe-) ribose, which confer nuclease resistance. However, these modifications typically must be introduced into natural DNA or RNA aptamers after selection by SELEX, and trial and error is necessary to find substitutions that do not compromise the binding activity of the selected molecule. Described herein are methods to overcome these challenges.

### BRIEF SUMMARY

In one aspect, a method for selecting fully modified RNA aptamers that bind to a target molecule without reverse transcription, comprises:
providing a first pool of oligonucleotide complexes that each comprise a ds-DNA molecule tethered to a fully modified RNA molecule, wherein the ds-DNA molecule comprises a coding strand and a complementary non-coding strand which encodes the RNA molecule;
exposing the first pool of oligonucleotide complexes to the target molecule and allowing the fully modified RNA molecules to bind the target molecule to provide a pool of selected oligonucleotide complexes comprising selected fully modified RNA aptamers which bind the target molecule;
amplifying the tethered ds-DNA molecules in the pool of selected oligonucleotide complexes using an affinity labeled forward primer and an unlabeled reverse primer to provide amplified affinity labeled ds-DNA molecules, wherein the affinity labeled forward primer binds a primer binding site near the 3′ end of the coding strand and the unlabeled reverse primer binds a primer binding site near the 3′ end of the non-coding strand, and wherein the affinity labeled ds-DNA molecules comprise an affinity labeled strand;
contacting the amplified affinity labeled ds-DNA molecules with an affinity label-binding molecule and selecting amplified affinity labeled ds-DNA molecules which bind the affinity label-binding molecule to provide affinity label-binding ds-DNA molecules; and
isolating the non-affinity labeled strand from the affinity label-binding ds-DNA molecules to provide a first amplified single-stranded DNA comprising a template for the selected fully modified RNA aptamers,
wherein a fully modified RNA molecule comprises 2′-fluoro-nucleotides, 2′-methoxy-nucleotides, or a combination of 2′-fluoro-nucleotides and 2′-methoxy-nucleotides, and no un-modified nucleotides.

In another aspect, a method of making a selection library comprises
providing a single-stranded DNA comprising a random library template flanked by 3′ and 5′ constant primer regions;
annealing the 3′ constant primer region of the single-stranded DNA to a phosphorylated overhanging regeneration primer and adding dNTPs and a first DNA polymerase to provide a bidirectionally extended duplex DNA having a phosphorylated stand and an unphosphorylated strand;
digesting the phosphorylated strand with an exonuclease to provide the unphosphorylated strand, wherein the unphosphorylated strand is a single-stranded hairpin template comprising a 3′ hairpin, the random library template, and the 5′ constant primer region;
extending the hairpin template using a second DNA polymerase and either all 2′F-NTPs and/or 2′-methoxy NTPS to provide a modified randomized nucleotide stand complementary to the random library template and the 5′ constant primer region and provide a double stranded hairpin structure; and
displacing the modified randomized nucleotide strand of the double stranded hairpin structure by treatment with a hairpin primer complementary to the 3′ hairpin sequence and extending the hairpin primer with a third DNA polymerase to provide the selection library containing the modified randomized nucleotide strand tethered to a double stranded DNA molecule comprising a coding strand and a complementary non-coding strand which encodes the RNA molecule, wherein the second DNA polymerase incorporates 2′-fluoro-nucleotides, 2′-methoxy-nucleotides, or a combination of 2′-fluoro-nucleotides and 2′-methoxy-nucleotides.

### BRIEF DESCRIPTION OF THE DRAWINGS

FIG. 1 shows the SELection with Modified Aptamers (SELMA) construct containing a PCR amplifiable DNA genotype that is covalently linked to a phenotype strand containing XNA and/or 5-ethynyluridine modified by click chemistry.

FIGS. 2-4 illustrate SELMA library construction.

FIG. 2 shows three enzymatic reactions used for installation of the hairpin to form DNA template Form D via Forms A-C.

FIG. 3 shows hairpin extension to prepare Form E from Form D.

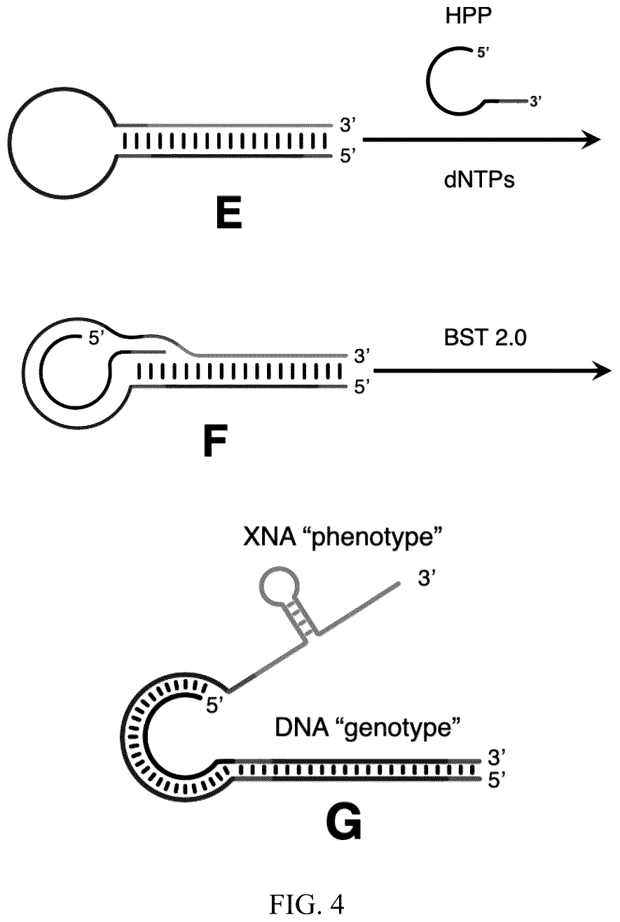

FIG. 4 shows stand displacement to prepare Forms F and G.

FIG. 5 shows the SFM4-3 hairpin extensions on various clones at 50° C. and 70° C. Extension progress after 30 minutes at 50° C. or 70° C. visualized on 10% Urea PAGE gel. SM=starting material. Clone sequences are presented in Table 1.

FIG. 6 shows extension temperature optimization of KOD DGLNK. 2 μL samples were removed and added to 8M Urea at different time points to evaluate KOD DGLNK's activity at 60° C. and 75° C. Full conversion to the final product (Form E) was observed at 30 minutes during the 75° C. but not observed after 180 minutes at 60° C.

FIG. 7 is a scheme of a click comparison between SFM4-3 and Therminator™ extension products done by a CuAAC with Man₉-cyclohexyl azide.

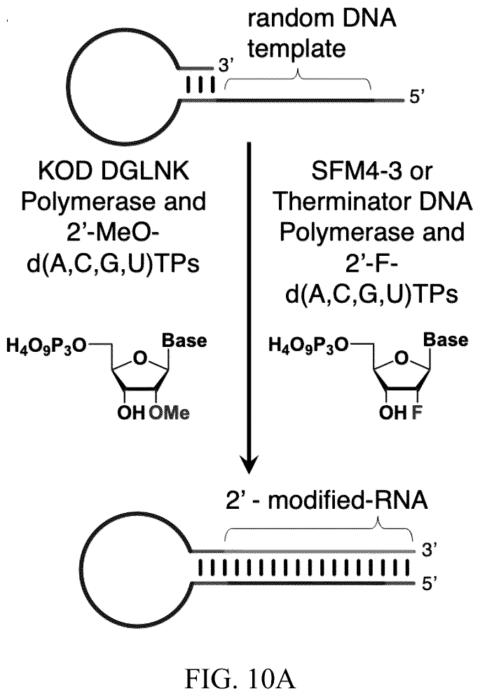

FIG. 8 shows complete PAGE of mock NheI selection gels. Random libraries produced by KOD DGLNK (with MeO-RNA) and Therminator™ DNA polymerase (with F-RNA) were spiked with NheI restriction sequence and subjected to 1 round of selection with a biotinylated oligo complementary to that sequence. (+) and (−) indicate samples with and without NheI treatment. Group 1=pure NheI sequence; Group 2=1000:1 mixture of random and NheI sequence before selection; Group 3=random:NheI mixture after selection; Group 4=random library alone; Group 5=random library alone; Group 6=1000:1 mixture of random and NheI sequence before selection; Group 7=1000:1 mixture of random and NheI sequence after selection; Group 8=pure NheI sequence.

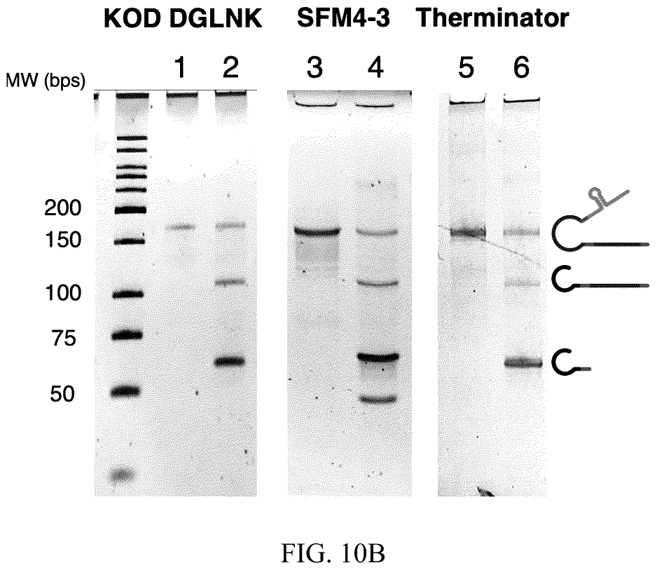

FIG. 9 shows a schematic of XNA SELMA Library build.

FIGS. 10 A and B show formation of double stranded hairpin and subsequent strand displacement. 10A) Scheme of hairpin extension and strand displacement. 10B) Denaturing PAGE gel of extended (lanes 1, 3 and 5) and displaced (lanes 2, 4, and 6) hairpin made with KOD DGLNK and MeO-RNA (lanes 1 and 2), SFM4-3 and F-RNA (lanes 3 and 4) and Therminator™ and F-RNA (lanes 5 and 6). The band below 50 bp in lane 4 is the NEB WarmStart® aptamer.

FIGS. 11A and B show compatibility of each enzyme with the SELMA workflow. 11A) NheI selection construct design and enrichment test. B=biotin; 11B) Gel analysis of NheI-digested templates from selections. NheI-treated samples are indicated with a (+). Group 1=random library post selection, Group 2=1:1000 NheI template:random library template pre selection, Group 3=1:1000 NheI template:random library template post selection, Group 4=NheI template only post selection. Groups 1-4 were prepared with SFM4-3. Groups 5 and 6 are post-selection libraries analogous to Group 3 but made with KOD DGLNK/2′-MeO-NTPs Therminator polymerase/2′-F-NTPs, respectively. Complete gels corresponding to Groups 5 and 6 can be found in FIG. 9

FIGS. 12A and B show generation of alkyne-containing F-RNA/DNA duplex, CuAAC modification with Man₉-azide glycan, and gel electrophoresis. 12A shows a schematic of the synthesis. 12B shows gel electrophoresis. Lanes 1 and 3=product of primer extension with Therminator™ and SFM4-3 respectively, in the presence of 2′-F(A,G,C,EdU) triphosphates. Lanes 2 and 4=Therminator™ and SFM4-3-derived oligos after click functionalization to attach Man₉ glycan.

The above-described and other features will be appreciated and understood by those skilled in the art from the following detailed description, drawings, and appended claims.

### DETAILED DESCRIPTION

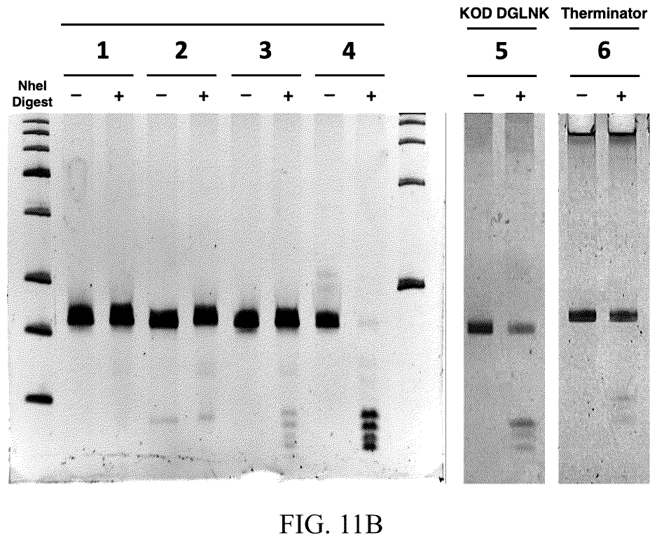

Described herein is a method for incorporating XNA throughout the selection process. A solution to this problem is the SELection of Modified Aptamers (SELMA) method (illustrated in FIG. 10) which allows for extensive modifications, not only of ribose, but of the nucleobase (e.g., oligosaccharide attachment) and includes a variant that can use 2′-F pyrimidine NTPs throughout the selection, for example. In SELMA, PCR and/or RT-PCR of modified oligonucleotides is circumvented by tethering an unmodified, PCR-amplifiable DNA copy to each XNA sequence in the library.

However, this technique has not yet been adapted to selections with libraries containing the 2′-F modification on all four nucleotides, which would require an RNA or DNA polymerase capable of incorporating them. Although several polymerases are known to accommodate ribose-modified NTPs, the activity and fidelity of these enzymes remains poorly understood and difficult to assess by traditional sequencing methods. To this end, three different polymerases capable of incorporating 2′-modified XNA have been incorporated into the SELMA protocol. Therminator™ DNA polymerase is a commercially available mutant of 9° N DNA polymerase that is able to incorporate a variety of non-canonical nucleic acids including 2′-F-modified NTPs (2′-F-NTPs). SFM4-3, which also incorporates 2′-F-NTPs, is a variant of the Stoffel fragment of TAQ DNA polymerase developed using directed evolution. KOD DGLNK is a mutant KOD polymerase that accepts both LNA and 2′-OMe-modified nucleotides.

Described herein is a method for evolving fully-fluorinated or fully-methoxy RNA aptamers that bind a target, e.g., a protein of interest. In the system, a DNA library hairpin that has single-stranded extension of random sequences followed by an adenosine-free constant region at the 5′ end is subjected to polymerase extension at the 3′ end of the hairpin, which serves as a primer. The extension reaction is done in the presence of 2′-fluoro-GTP, 2′-fluoro-ATP, 2′-fluoro-CTP and 2′-fluoro-EdUTP (or 2′-fluoro-UTP) using Stoffel fragment mutant 4-3 or Therminator polymerase, or all 2′-methoxy-NTPs such as 2′methoxy-EdUTP using KOD DGLNK polymerase. The reaction results in formation of a complete hairpin structure that is a hybrid of DNA and 2′-fluoro or 2′-methoxy RNA with or without ethynyl bases. The alkynes on 2′-fluoro-EdUTP bases are glycosylated by click reaction. In the next step, polymerase extension with a DNA primer that anneals to a loop region of the hairpin is performed. As a result, glycan-attached 2′-fluoro RNA is displaced from the annealed DNA but still flanked with the generated dsDNA, which serves as a genetic code. The library is mixed with an immobilized target to select binders, and the DNA of the selected binders are amplified by PCR to create a library of the next selection round. The cycle is repeated until target-binders are identified.

As described in detail herein, several modifications to the SELMA methodology allow for the incorporation and in vitro selection of fully 2′-modified XNA without the need for reverse transcription or post-SELEX medicinal chemistry optimization to incorporate nuclease resistant modifications. Additionally, it is demonstrated that fully 2′-modified XNA SELMA constructs can be successfully enriched towards a known target. This expansion of SELMA capabilities will simplify discovery of aptamers that could be directly used in the presence of nucleases. The 2′F-RNA can be chemically modified by CuAAC, and Therminator™ is less prone to misincorporation of 2′-fluoro-EUTP than SFM4-3.

U.S. Pat. Nos. 11,268,099; 10,125,162; 11,732,256; 10,780,150; and 11,976,273 are incorporated by reference herein for their disclosures of the SELMA method and variations thereof.

In an aspect, a method for selecting fully modified RNA aptamers that bind to a target molecule without reverse transcription comprises
providing a first pool of oligonucleotide complexes that each comprise a ds-DNA molecule tethered to a fully modified RNA molecule, wherein the ds-DNA molecule comprises a coding strand and a complementary non-coding strand which encodes the RNA molecule;
exposing the first pool of oligonucleotide complexes to the target molecule and allowing the fully modified RNA molecules to bind the target molecule to provide a pool of selected oligonucleotide complexes comprising selected fully modified RNA aptamers which bind the target molecule;
amplifying the tethered ds-DNA molecules in the pool of selected oligonucleotide complexes using an affinity labeled forward primer and an unlabeled reverse primer to provide amplified affinity labeled ds-DNA molecules, wherein the affinity labeled forward primer binds a primer binding site near the 3′ end of the coding strand and the unlabeled reverse primer binds a primer binding site near the 3′ end of the non-coding strand, and wherein the affinity labeled ds-DNA molecules comprise an affinity labeled strand;
contacting the amplified affinity labeled ds-DNA molecules with an affinity label-binding molecule and selecting amplified affinity labeled ds-DNA molecules which bind the affinity label-binding molecule to provide affinity label-binding ds-DNA molecules; and
isolating the non-affinity labeled strand from the affinity label-binding ds-DNA molecules to provide a first amplified single-stranded DNA comprising a template for the selected fully modified RNA aptamers,
wherein a fully modified RNA molecule comprises 2′-fluoro-nucleotides, 2′-methoxy-nucleotides, or a combination of 2′-fluoro-nucleotides and 2′-methoxy-nucleotides, and no un-modified nucleotides.

In an aspect, the first pool of oligonucleotide complexes comprises a selection library made by the methods described herein.

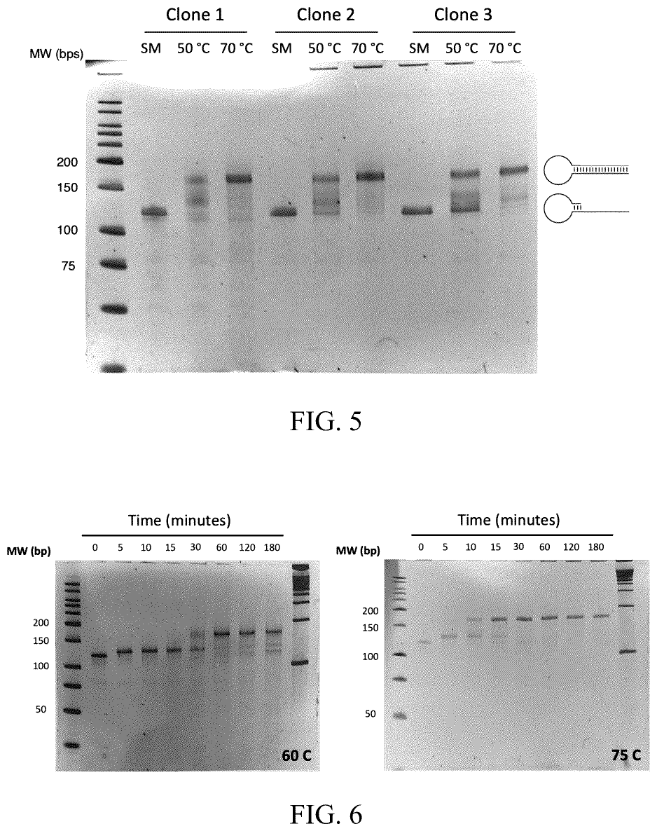

The first step of the method comprises providing a first pool of oligonucleotide complexes that each comprise a ds-DNA molecule tethered to a fully modified RNA molecule, wherein the ds-DNA molecule comprises a coding strand and a complementary non-coding strand which encode the RNA molecule. The pool of oligonucleotide complexes corresponds to Form G in FIG. 4, for example. The XNA “phenotype” is a fully modified RNA, and the ds-DNA molecule provides the “DNA genotype” for the RNA. The oligonucleotide complexes preferably include a forward primer binding site and a reverse primer binding site for amplification of the selected oligonucleotide complexes.

As used herein, a fully modified RNA molecule comprises 2′-fluoro-nucleotides, 2′-methoxy-nucleotides, or a combination of 2′-fluoro-nucleotides and 2′-methoxy-nucleotides, and no un-modified nucleotides. That is, each nucleotide of the fully modified RNA molecule is modified by 2′-fluoro or 2′-methoxy. Prior to the experiments provided herein, it was not known if it would be possible to synthesize and amplify fully modified RNA molecules, as RNA polymerization and amplification typically have limited tolerance for modified nucleoside triphosphates.

The first pool of oligonucleotide complexes is exposed to the target molecule and the modified RNA molecules are allowed to bind the target molecule to provide a pool of selected oligonucleotide complexes comprising selected fully modified RNA aptamers which bind the target molecule. This is a round of selection. The RNA aptamers possess specificity in binding to a particular target molecule, and they non-covalently bind their target molecule through an interaction such as an ion-ion force, dipole-dipole force, hydrogen bond, van der Waals force, electrostatic interaction, stacking interaction or any combination of these interactions. Aptamers that bind to their target with low nM affinity, or sub-nM affinity, are particularly desirable, and aptamers that possess modified RNA to promote, e.g., stability and resistance to degradation, are also desirable.

The target molecule for the methods described herein is not particularly limited and can be a small molecule or a biomolecule such as a polypeptide. Exemplary target molecules include targets include proteins (e.g., enzymes, receptors, antibodies, protein toxins), peptides, small molecules (e.g., drugs, toxins, amino acids, carbohydrates,), and the like.

The next step comprises amplifying the tethered ds-DNA molecules in the pool of selected oligonucleotide complexes using an affinity labeled primer to provide amplified affinity labeled ds-DNA molecules, wherein the affinity labeled ds-DNA molecules comprise an affinity labeled strand. An exemplary affinity labeled primer is a biotinylated primer.

During amplification of the ds-DNA molecules from the selected oligonucleotide complexes, an affinity labeled forward primer is used and binds to a primer binding site near the 3′ end of the template, i.e., coding, strand. A reverse primer which is not affinity tagged binds to a primer binding site near the 3′ end of the non-coding strand.

The next step includes contacting the amplified affinity labeled ds-DNA molecules with an affinity label-binding molecule and selecting amplified affinity labeled ds-DNA molecules which bind the affinity label-binding molecule to provide affinity labeled-binding ds-DNA molecules. Exemplary biotin binding molecules include streptavidin, NeutrAvidin, and avidin.

In the next step, the affinity labeled strand is removed from the affinity label-binding ds-DNA molecules to provide a first amplified single-stranded DNA comprising a template for the selected fully modified RNA aptamers. The affinity labeled strand can be removed using magnetic beads attached to the affinity label-binding molecule, for example.

Exemplary affinity labels include biotin, Step-Tag® which binds Strep-Tactin®, His-tags which bind Ni-NTA, GST tags which bind glutathione resin, and the like.

Unlike traditional SELEX where the RNA is typically supported on a solid support, i.e., as a partitioning device, the present method involves retaining the RNA tethered to the ds-DNA containing the template. As such, there is no need to use reverse transcription to regenerate the template, because the selected RNA and its template remain tethered throughout each round of selection. In other words, each round of selection for RNA binding to its target necessarily involves selection of the tethered template DNA. This enhances the efficiency of SELEX by avoiding unnecessary steps.

In an aspect, the method can comprise further rounds of selection. Thus, in a specific aspect, the method can further comprise providing the first amplified single-stranded DNA comprising a template for the selected fully modified RNA aptamers, and preparing a second pool of oligonucleotide complexes that each comprise a ds-DNA molecule tethered to a fully modified RNA molecule, wherein the ds-DNA molecule comprises a coding strand and a complementary non-coding strand which encode the RNA molecule, and repeating the exposing, selecting, amplifying, contacting and isolating steps to provide a second amplified single-stranded DNA comprising a template for the selected fully modified RNA aptamers.

In another aspect, the 2′-fluoro-nucleotides and/or 2′-methoxy-nucleotides can comprise one or more alkyne-containing nucleotides (e.g., C8 octadiynyl substituent, 5-ethynyl-2′-deoxyuridine). In an aspect, one or more U, A, G or C nucleotides is alkyne-substituted.

In another aspect, the fully modified RNA molecule containing an alkyne-substituted nucleotide is glycosylated. In the SELMA process described above, click-chemistry can be used to introduce desired modification, e.g., glycosylation, onto modified nucleotides prior to selection. Glycosylation of the library can be done using click chemistry.

In another aspect, the fully modified RNA molecule is covalently linked to a branched or unbranched oligosaccharide. Linking of an oligosaccharide can also be done using click chemistry.

Methods of making a selection library for selection of fully modified RNAs is also described herein.

In an aspect, a selection library, e.g., the first pool of oligonucleotide complexes, is made by a method comprising
providing a first single-stranded DNA comprising a random library template flanked by 3′ and 5′ constant primer regions;
annealing the 3′ constant primer region of the single-stranded DNA to a phosphorylated overhanging regeneration primer and adding dNTPs and a first DNA polymerase to provide a bidirectionally extended duplex DNA having a phosphorylated stand and an unphosphorylated strand;
digesting the phosphorylated strand with an exonuclease to provide the unphosphorylated strand, wherein the unphosphorylated strand is a single-stranded hairpin template comprising a 3′ hairpin, the random library template, and the 5′ constant primer region;
extending the hairpin template using a second DNA polymerase (e.g., Therminator DNA polymerase or KOD DGLNK) and either all 2′-F-NTPs, all 2′-methoxy-NTPS, or all a combination of 2′-F-NTPs and 2′-methoxy-NTPS, to provide a modified randomized nucleotide strand complementary to the random library template and the 5′ constant primer region and provide a double stranded hairpin structure; and
displacing the modified randomized nucleotide strand of the double stranded hairpin structure by treatment with a hairpin primer complementary to the 3′ hairpin sequence and extending the hairpin primer with a third DNA polymerase to provide the selection library containing the modified randomized nucleotide strand tethered to a double stranded DNA molecule comprising a coding strand and a complementary non-coding strand which encodes the RNA molecule.

The Therminator™ DNA polymerase is an engineered Thermococcus sp. 9N-7 DNA polymerase with an A485L mutation that allows it to efficiently incorporate unconventional substrates such as 2′-F-NTPs.

The KOD DGLNK is a Thermococcus kodakarensis KOD1 polymerase with N210D/Y409G/A485L/D614N/E664K mutations as known in the art. KOD DGLNK can incorporate 2′-methoxy-NTPs.

In an aspect, the exonuclease comprises a first exonuclease to digest primer and a second exonuclease to digest the phosphorylated strand.

In an aspect, the first amplified single-stranded DNA comprising the template for the selected fully modified RNA aptamers is regenerated to form a second single-stranded DNA comprising a second random library template flanked by 3′ and 5′ constant primer regions.

Also included are fully modified RNA aptamers that bind to a target molecule selected by the method described herein.

Further included is a fully modified RNA aptamer that binds to a target molecule selected by the method described herein, wherein the fully modified RNA aptamer is conjugated to an amplified single-stranded DNA comprising a template for the RNA.

Yet further included is a fully modified RNA aptamer that binds to a target molecule selected by the method described herein, wherein the fully modified RNA aptamer is not conjugated to an amplified single-stranded DNA comprising a template for the RNA.

The invention is further illustrated by the following non-limiting examples.

### EXAMPLES

### Methods

Materials: DNA oligonucleotides were purchased from Integrated DNA Technologies (Coralville, IA) and purified using denaturing polyacrylamide gel electrophoresis (PAGE) according to the manufacturer's recommendations. All sequences purchased for the experiments herein can be found in Table S1. Enzymes including BST 2.0 WarmStart® DNA polymerase, BST 2.0 DNA polymerase, Exonuclease I, Lambda Exonuclease, Therminator™ DNA Polymerase, and Phusion™ DNA Polymerase were purchased from New England Biolabs (Ipswich, MA). Additionally, dNTPs were purchased from New England Biolabs and all 2′-F-NTPs except 2′-fluoro-ethynyluridine (2′-F-EdUTP) were purchased from TriLink BioTechnologies (San Diego, CA). 2′-F-EdUTP was synthesized in-house using our published route. Hydrophilic streptavidin M270-Dynabeads™ were purchased from Invitrogen (Waltham, MA). Sybr™ Gold nucleic acid gel stain was purchased from Life Technologies. CentriSep™ 10 and 20 spin columns were purchased from Princeton Separations (Princeton, NJ). All other reagents were purchased from National Diagnostics, Sigma-Aldrich, Acros Organics, New England Biolabs, or Fisher and used without further purification unless otherwise noted.

TABLE 1
Oligonucleotide sequences

| Name | Sequence | SEQ ID NO: |
|---|---|---|
| Library Template | CTTGTCGTCTCCTGTGTGCTTNNNNNN | 1 |
| | NNNNNNNNNNNNNNNNNNNCCCGTA | |
| | CCCGTTAAAACTCCACCTCATAACCG | |
| | CA | |
| Regeneration | /5Phos/CCCGTACCCGAATATAAAATAA | 2 |
| Primer | AAATATAAAATATAAAATTGCGGTTA | |
| | TGAGGTGGAGTT | |
| Hairpin Poison | TAAAATAAAAATATAAAATATAAAAT | 3 |
| Primer | TGCGGTTATGAGGTGGAGTTTTAACG | |
| | GGTACGGG | |
| Biotin Forward | /5Biosg/TGCGGTTATGAGGTGGAGTT | 4 |
| Primer | | |
| Reverse Primer | CTTGTCGTCTCCTGTGTGCTT | 5 |
| NheI Template | CTTGTCGTCTCCTGTGTGCTTCGTTCTT | 6 |
| | ATGTTCTCACTCGCTAGCCCCGTACCC | |
| | GTTAAAACTCCACCTCATAACCGCA | |
| NheI Selection | /5BiotinTEG/TTCTCACTCGCTAGC | 7 |
| Primer | | |
| Clone 1 | CTTGTCGTCTCCTGTGTGCTTTATCCG | 8 |
| | TAGGTTGCACCGTGGGTCTCCCGTACC | |
| | CGTTAAAACTCCACCTCATAACCGCA | |
| Clone 2 | CTTGTCGTCTCCTGTGTGCTTTGACCC | 9 |
| | ACGGTGCAACCTACGGATACCCGTAC | |
| | CCGTTAAAACTCCACCTCATAACCGC | |
| | A | |
| Clone 3 | CTTGTCGTCTCCTGTGTGCTTCATAGG | 10 |
| | GAACCCAGGTGATTGGGGCCCCGTAC | |
| | CCGTTAAAACTCCACCTCATAACCGC | |
| | A | |
| Click test | CTTGTCGTCTCCTGTGTGCTTTATCCG | 11 |
| template | TAGGTTGCACCGTGGGTCTCCCGTACC | |
| | CG | |
| Click test primer | CGGGTACGGGAGACC | 12 |

TABLE 2

| Buffer name | 1X Component Concentrations |
|---|---|
| Hairpin Storage Buffer | 2 mM MgSO₄ and 10 mM Tris-HCl pH 7.5 |
| Click Buffer | 2 mM MgSO₄ and 10 mM HEPES pH 8 |
| Streptavidin Bead Buffer | 1M NaCl, 0.5 mM EDTA, 5 mM Tris-HCl |
| | pH 7.5 and 0.1% Tween ® 20 |
| Binding Buffer with | 150 mM NaCl, 2 mM MgSO₄, 20 mM Tris- |
| Tween (BBT) | HCl pH 7.5 and 0.1% Tween ® 20 |
| SFM Wash Buffer | 50 mM NaH₂PO₄, 300 mM NaCl, 20 mM |
| | Imidazole, pH 8.0 |
| Equilibration Buffer | 10 mM (NH₄)₂SO₄, 100 mM NaCl, 1 mM |
| | DTT, 10% glycerol |
| KOD Exchange Buffer | 1 mM DTT, 50 mM Tris-HCl pH 8, 0.1% |
| | Nonidet ™ P-40, 0.1% Tween ® 20, 10% |
| | glycerol |
| KOD Storage Buffer | 1 mM DTT, 50 mM Tris-HCl pH 8, 0.1% |
| | Nonidet ™ P-40, 0.1% Tween ® 20, 90% |
| | glycerol |
| KOD Buffer #2 | 6 mM (NH₄)₂SO₄, 10 mM KCl, 120 mM |
| | Tris-HCl pH 8.8, 0.1% Triton ™ X-100, |
| | 0.001% BSA |

### Enzyme Expression and Purification

SFM4-3 Expression and Purification: Plasmid for the SFM4-3 polymerase was obtained from the Romesburg lab (Scripps Research Institute) and competent BL21 (DE3) pLysS cells were purchased from Invitrogen. Pure plasmid was transformed into the competent cells through heat shock according to manufacturer's instructions for toxic gene expression. Transformed cells were then diluted into prewarmed SOC broth and incubated at 37° C. for 1 hour. These cells were then inoculated into 1 L of 2×YT growth media and left to grow at 37° C. After reaching an OD of 0.6, transcription was induced by adding IPTG to a concentration of 0.4 mM and the incubation temperature lowered to 30° C. After 18 hours, the cells were pelleted at 4000×g for 20 minutes at 4° C. The supernatant was poured off and pelleted cells were resuspended in 25 mL of 1×PBS+1× BugBuster® Protein Extraction Reagent (Novagen). This suspension was incubated at 70° C. for 30 minutes. Cell debris was pelleted at 10,000×g for 45 minutes at 4° C. and the supernatant was then tumbled with 1 mL of Ni-NTA resin for 1 hr at 4° C. The slurry was then applied to an EconoPac® column (BioRad) and the flow through collected. The resin was washed 3 times with 5 mL of the SFM Wash Buffer. SFM4-3 was eluted in 3 subsequent 5 mL washes of 50 mM, 100 mM, and 250 mM imidazole+0.5 mM B-ME respectively and collected in 1 mL fractions. SDS-PAGE was used to determine which fractions contained the polymerase and these were subsequently pooled. The collected fractions were then dialyzed in a 20K cassette with 50 mM Tris-HCl (pH 8.5) and 0.5 mM EDTA overnight. After dialysis, the enzyme was concentrated to roughly 150 μL using 30 kDa Vivaspin™ columns and 150 μL of glycerol was added for 50% glycerol stock storage. For subsequent use, the enzyme was diluted to the desired activity which was to extend 8 picomoles of single stranded hairpin in 30 minutes. This test and dilution was done for each aliquot of the enzyme between expressions.

KOD DGLNK Expression and Purification: Plasmid for the KOD DGLNK polymerase was obtained from the Obika lab (Osaka University) and was transformed into BL21 (DE3) pLysS cells according to the manufacturer's instructions. A 250 mL culture was grown in 2×YT media containing 100 g/mL kanamycin and 34 μg/mL chloramphenicol at 37° C. to an OD of 0.6 before inducing with 0.4 mM IPTG. Cells were pelleted at 4000×g for 20 minutes at 4° C. Pelleted cells were resuspended in 20 mL 1× BugBuster® Protein Extraction Reagent (Novagen) in PBS and incubated at 70° C. for 30 minutes. Cell debris was pelleted by centrifugation at 10000×g for 40 minutes at 4° C., and the supernatant was filtered with a 0.22 μm cellulose acetate filter before FPLC purification.

For FPLC purification of KOD DGLNK a 1 mL HiTrap™ Heparin HP column (Cytiva) was used. First the column was equilibrated with 10 mL of Equilibration Buffer. Next 8-10 mL of crude protein was applied onto the column using 3-4 injections of a 2 mL sample injection loop. A linear gradient of 0.1-1.5 M NaCl was applied for 20 minutes at a flow rate of 1 mL/minute, and 1 mL fractions collected. SDS-PAGE was used to identify the fractions containing KOD DGLNK. These were combined and buffer exchanged into KOD Exchange Buffer using a 30 kDa MWCO filter to 150 μL. The concentrated protein was combined with an equal volume of KOD Storage Buffer and stored at −20° C. For subsequent use, the enzyme was diluted based to the desired activity which was to extend 8 picomoles of single stranded hairpin in 30 minutes. This test and dilution was done for each aliquot of the enzyme between expressions.

### SELMA Library Construction

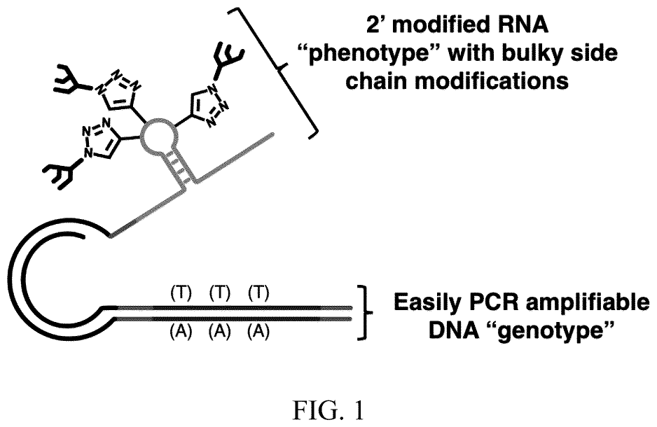

Installation of the hairpin involves three enzymatic reactions illustrated in FIG. 1. Starting with 50 pmol of a single stranded 80 base pair template sequence (Form A) (either a single sequence or library), Thermopol® Buffer (1× final concentration), 100 pmol of Regeneration Primer, and water were added to a final volume of 100 μL in a PCR tube. This mixture was heated to 95° C. for 1 minute, cooled to 45° C. for 1 minute, and then left at room temperature. dNTPs (200 μM each final concentration) and 2 U of BST 2.0 WarmStart® DNA Polymerase were added to the mixture and then heated to 60° C. for 10 minutes to create a double stranded product with one strand phosphate labeled (Form C), then buffer exchanged into water using CentriSep™-20 columns (Princeton Separations). Next, 1 U of Exonuclease 1 was added with Exonuclease 1 Buffer (1× final concentration) and the mixture heated to 37° C. for 30 minutes followed by 80° C. for 20 minutes. This digest was then buffer exchanged into Hairpin Storage Buffer using CentriSep™-20 columns (Princeton Separations). Finally, 1 U of Lambda Exonuclease was added with Lambda Exonuclease Buffer (1× final concentration) and the mixture heated to 37° C. for 30 minutes followed by 75° C. for 10 minutes to generate Form D, and buffer exchanged into water using CentriSep™-20 columns (Princeton Separations).

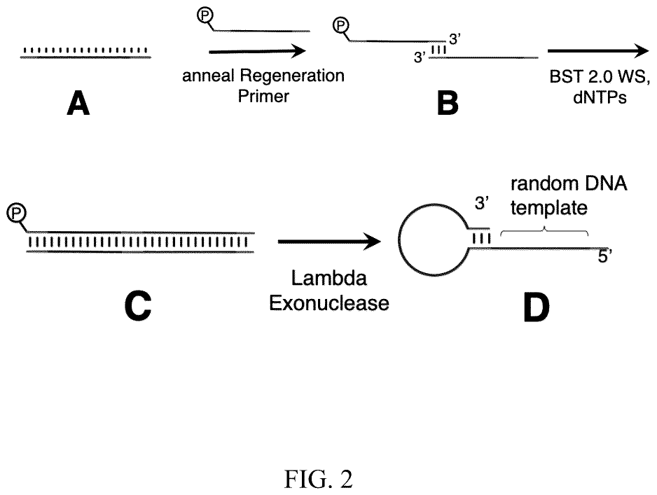

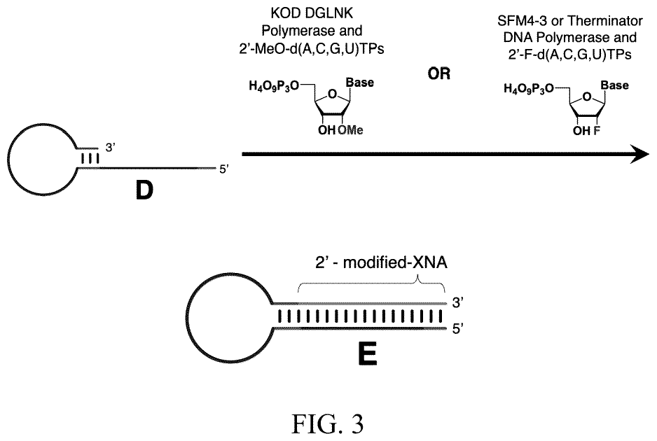

After creation of single-stranded hairpin by the steps above, extension was done with 2′ modified RNA using one of three enzymes: SFM4-3, Therminator™, KOD DGLNK as shown in FIG. 2.

Standard SFM4-3 hairpin extension reactions included 1× Taq Buffer (NEB), 0.2 mM single stranded hairpin (Form D), 2 mM supplemental MgCl₂, 1 mM MnCl₂, and 0.4 mM 2′-F-NTPs. Optimal SFM4-3 concentration varied between enzyme purifications and was determined experimentally as the minimal amount of enzyme required to extend 8 pmol of hairpin in 30 minutes. Hairpin template was heated to 95° C. for 1 minute and cooled to 45° C. for 5 minutes to promote self-priming prior to the addition of salts, NTPs, and SFM4-3. The complete reaction mixture was then incubated at 70° C. for 30 minutes to generate Form E.

Standard Therminator™ hairpin extension reactions included 1× Thermopol® Buffer (NEB), 0.2 mM single stranded hairpin (Form D), 0.4 mM 2′-F-NTP mix, and 2 U of Therminator™ polymerase (NEB). Hairpin template was heated to 95° C. for 1 minute and cooled to 45° C. for 5 minutes to promote self-priming prior to the addition of NTPs, and Therminator™. The complete reaction mixture was then incubated at 60° C. for 10 minutes to generate Form E.

Standard KOD DGLNK hairpin extension reactions included 1×KOD Buffer #2, 0.2 mM single stranded hairpin (Form D), 1 mM MgSO₄, and 0.2 mM 2′-OMe-NTP mix. Optimal KOD DGLNK concentration varied between enzyme purifications and was determined experimentally as the minimal amount of enzyme required to extend 8 pmol of hairpin in 30 minutes. Hairpin template was heated to 95° C. for 1 minute and cooled to 45° C. for 5 minutes to promote self-priming prior to the addition of NTPs, and KOD DGLNK. The complete reaction mixture was then incubated at 75° C. for 1 hour to generate Form E.

The hairpin extension, regardless of enzyme used, was followed by buffer exchanging into Hairpin Storage Buffer using CentriSep™-10 columns (Princeton Separations).

Next, as shown in FIG. 4, the modified RNA was strand displaced using the hairpin poison primer (HPP; also called a hairpin primer herein) and BST 2.0 DNA polymerase. Typical reactions included 0.8 μM of product E, 1× Thermopol® Buffer (NEB), 0.16 μM HPP, and 0.6 mM dNTPs in a final volume of 50 μL. The reaction was heated to 95° C. and cooled at 0.3° C. per minute to 25° C. to generate Form F. 8 U of BST 2.0 DNA polymerase were then added and the reaction is incubated at 55° C. for 40 minutes to produce Form G. This material was then buffer exchanged into Hairpin Storage Buffer using CentriSep™-20 columns (Princeton Separations).

When strand displacement was performed using BST 2.0 WarmStart® DNA polymerase (BST 2.0 is inhibited by the “WarmStart®” aptamer, which binds the enzyme active site at cooler temperatures), we found that the NEB WarmStart® aptamer forms an isolable complex with our strand-displaced library. To demonstrate this, the strand displacement product generated using BST 2.0 WarmStart® Polymerase was run on an 8% native gel. Each band present was excised from the gel and crushed into small pieces. The DNA from each band was then extracted in 400 μL of 0.4 mM KOAc while rotating at room temperature overnight. The supernatant was concentrated with a speed vac to ~50 μL followed by buffer exchanging into water twice using CentriSep-20 columns (Princeton Separations) and then finally concentrated to ~20 μL. This concentrated DNA extract was then analyzed on a 10% denaturing urea PAGE gel and we were able to see that the extracted species contain the WarmStart® aptamer. Representative gel images are shown in FIG. 4. In the Native gel, the full construct band is indicated with a red box, all lower bands are composed of the HPP. The Warmstart® aptamer is apparent in the denaturing gel and indicated with a bracket.

The same procedure as described for FIG. 4 with BST 2.0 DNA polymerase that contained no WarmStart® aptamer to show that after gel excision no 40 bp aptamer remained (data not shown).

### SFM4-3 and KOD DGLNK Temperature Optimization

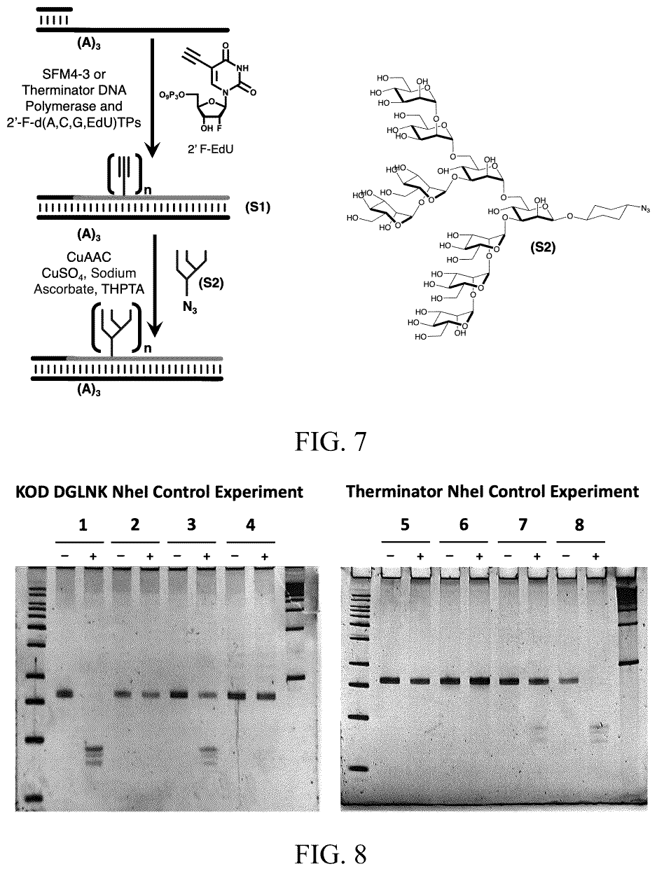

To optimize extension temperature for SFM4-3 and KOD DGLNK reactions, standard hairpin reactions were performed as described herein with the exception of varying extension temperatures. For SFM4-3, three sequences identified in previous SELMA selections were used as control templates. Extension progress was visualized on a 10% Urea gel run at 300V for 15 minutes (FIG. 5).

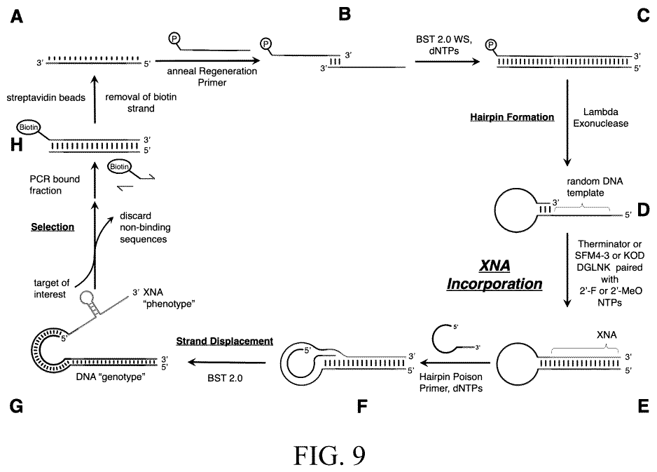

For KOD DGLNK, single stranded hairpin made from the library template was extended at various temperatures as shown in FIG. 6.

### SFM4-3 and Therminator Click Comparison

A click comparison between SFM4-3 and Therminator™ extension products was done by a CuAAC with Man₉-cyclohexyl azide, as previously reported in the art. To generate a clickable fluorinated RNA strand (S1), the Therminator™ and SFM4-3 extension protocols were completed using the Click test template and primer. After the extension the product was buffer exchanged into Click buffer using CentriSep™-10 columns (Princeton Separations). Tubes containing dry or degassed reaction components were prepared as follows: 202 nmol sodium ascorbate solution was placed in a capless 0.5 mL Eppendorf tube. Next, 90 nmol CuSO₄ and 108 nmol THPTA solutions were added to a second capless tube. Lastly, 5 pmol of the Therminator™ or SFM4-3 extension product of the click test primer and template and 50 nmol of Man₉-cyclohexyl-N₃ (S2) were placed in a third tube. All tubes were concentrated via speed-vac until the CuSO₄/THPTA and sodium ascorbate were dry and the library-sugar mixture was approximately 20 μL in volume. All tubes were then transferred to a 2-neck flask with N₂ gas flowing through. After increasing the flow of nitrogen gas, 5 μL of degassed water from a separate 2-neck flask was used to redissolve the sodium ascorbate. Next the dissolved sodium ascorbate was added to the tube with CuSO₄ and THPTA to reduce the Cu(II) to Cu(I). Subsequently, the mixture was added to the final tube of oligonucleotide-sugar mixture. The vessel was then flushed with nitrogen for 10 minutes before closing the septum while the reaction ran under slight N₂ pressure for 3 hours. The reaction was then diluted to 50 μL and buffer exchanged into MQ CentriSep™-10 columns (Princeton Separations).

### NheI Selection

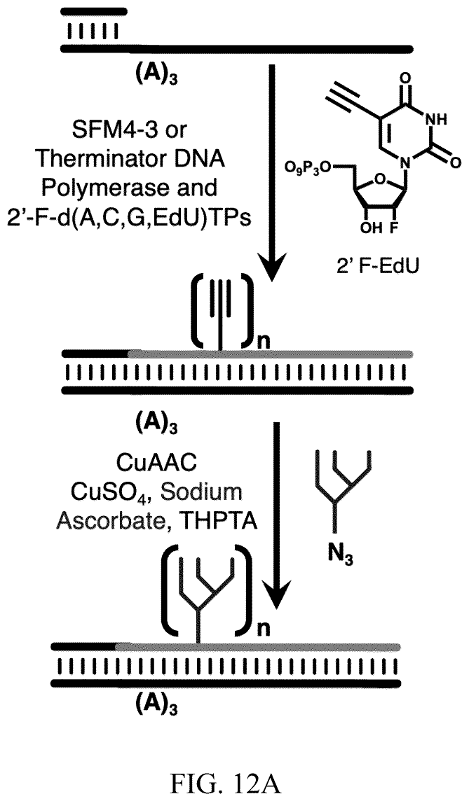

Briefly, 2 pmol of SELMA library form G was made, and 4 pmol of biotinylated NheI selection primer were diluted to a final volume of 50 μl in water and rotated for 10 minutes at room temperature. 0.1 mg streptavidin magnetic beads (Dynabeads™ M270) were then added, and the reaction was rotated for 20 minutes at room temperature. The supernatant was removed, and the beads were washed twice with Streptavidin Bead Buffer. Bound sequences were eluted by resuspending the beads in 50 μl 0.15 M NaOH for 4 minutes at room temperature. Supernatant was removed and neutralized with the addition of 5.5 μl 100 mM Tris and 6.5 μl of 1.25 M HOAc. Eluted sequences were used as PCR template, and the resulting PCR products were digested with NheI according to manufacturer's instructions. The results are shown in FIG. 11B.

### Example 1: XNA SELMA Library Build and Optimization

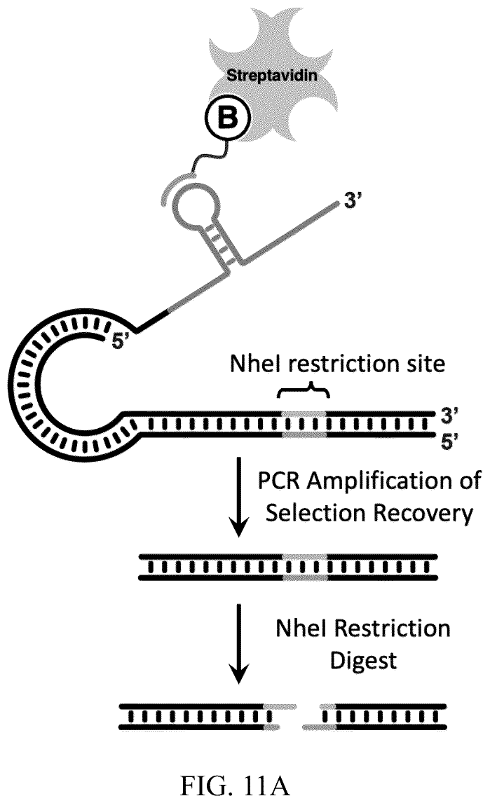

Commercially available Therminator™ was purchased and SFM4-3 and KOD DGLNK were expressed for generation of the ssXNA “phenotype” strand (G, FIG. 9). Library construction begins with a short ssDNA strand containing the random library template (A), which is annealed to a phosphorylated overhanging “regeneration” primer (B). Bidirectional DNA polymerase extension creates a longer duplex (C) and lambda exonuclease digestion of the phosphorylated strand results in a single stranded hairpin (D). The 3′ end of D is self-priming and can be extended with XNA using one of the three above enzymes to afford double stranded hairpin (E). The XNA strand is then displaced (F) by treatment with a “hairpin poison primer” (HPP) and its extension with BST 2.0 to generate Form G. Compared with the DNA/DNA duplex of our original SELMA method, this relatively long primer is required for the strand displacement of the stronger 2′-F-RNA-DNA interaction. The resulting Form G library contains modified ssXNA that can adopt a sequence-dependent fold and can undergo selection towards a target, while PCR amplification of the tethered dsDNA in the bound library fraction affords a new template in double stranded Form H. The use of the biotinylated primer in PCR allows for strand separation and a return to ssDNA library template (Form A).

For KOD DGLNK and SFM4-3, some optimization was done to characterize the behavior of each enzyme at various temperatures. Increasing the extension temperature from previously reported conditions resulted in more efficient extension and higher yields of Form E (FIG. 5,6). After determining the ideal extension conditions, each enzyme was used to form double stranded hairpin and subsequently strand displaced as shown in FIGS. 10A and B.

To demonstrate compatibility of each enzyme with the SELMA workflow, mock selections were performed in which a known target sequence of 2′-F- or 2′-OMe-RNA was selected from a random library by hybridization to a biotinylated complementary oligonucleotide (FIG. 11A). The target sequence contained an NheI restriction site, which enabled easy detection of the target sequence by the presence of four fragment bands after digestion with NheI (FIG. 11B, Group 4). This sequence was spiked at a ratio of 1:1000 into N₂₅ random libraries generated with 2′-F-RNA using Therminator™ or SFM4-3, or with 2′-OMe-RNA using KOD DGLNK. Prior to selection, the known sequence was not visible by gel analysis of the spiked libraries following NheI digest (e.g., FIG. 11B, Group 2). Each library was then subjected to one round of selection. Species that bound to the biotinylated complementary oligonucleotide were recovered using streptavidin magnetic beads. Bound material was then PCR amplified and digested with NheI, resulting in the expected four small fragment bands in all three selections (FIG. 11B, Groups 3, 5, 6). Thus, clear enrichment of the target sequence was observed after just one round of selection using libraries generated with each of the enzymes tested. Control selections containing only random library (FIG. 11B, Group 1) or only NheI (FIG. 11B, Group 4) sequence were also performed in parallel. The successful mock selection demonstrates the capability of the SELMA method to evolve a fully fluorinated or fully methoxy library and generate enrichment without the need for a reverse transcription step.

### Example 2: Confirm Chemical Functionalization of Libraries

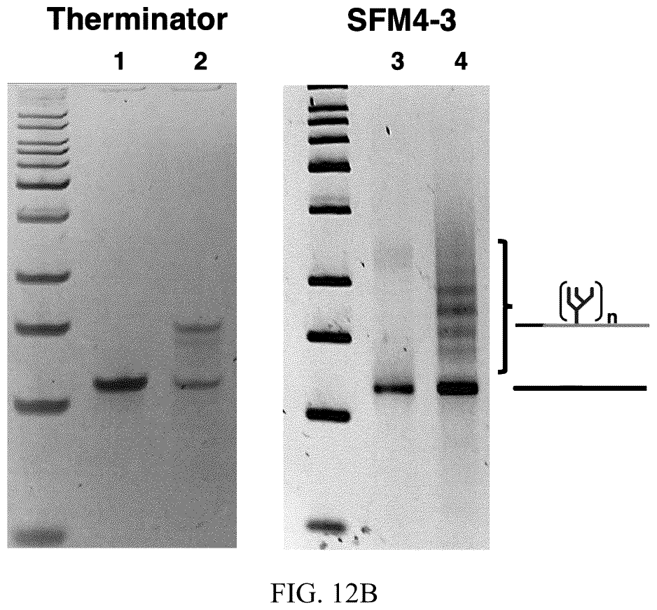

To investigate chemical functionalization of libraries, 2′-F-RNA either Therminator™ or SFM4-3 were used to generate an F-RNA strand containing three 5-ethynyluridine (2′-F-EdU) bases by primer extension against a 56-mer DNA template containing three adenines (FIG. 12A). Following CuAAC attachment of oligomannose (Man₉) glycan, the results were visualized by gel, in which the functionalized F-RNA strand migrates higher due to added glycans. When Therminator™ was used to generate the F-RNA strand, the corresponding glyco-F-RNA migrates primarily as one band, corresponding to addition of three glycans, with a small amount of a lower band corresponding to incomplete functionalization. However, when SFM4-3 was used to generate the F-RNA, the glyco-F-RNA is a series of bands, corresponding to different numbers of attached glycans in some cases exceeding three (FIG. 12B). These results suggested potential misincorporation of 2′-F-EdU by SFM4-3.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method for selecting fully modified RNA aptamers that bind to a target molecule without reverse transcription, comprising:
providing a first pool of oligonucleotide complexes that each comprise a ds-DNA molecule tethered to a fully modified RNA molecule, wherein the ds-DNA molecule comprises a coding strand and a complementary non-coding strand which encodes the RNA molecule;
exposing the first pool of oligonucleotide complexes to the target molecule and allowing the fully modified RNA molecules to bind the target molecule to provide a pool of selected oligonucleotide complexes comprising selected fully modified RNA aptamers which bind the target molecule;
amplifying the tethered ds-DNA molecules in the pool of selected oligonucleotide complexes using an affinity labeled forward primer and an unlabeled reverse primer to provide amplified affinity labeled ds-DNA molecules, wherein the affinity labeled forward primer binds a primer binding site near the 3′ end of the coding strand and the unlabeled reverse primer binds a primer binding site near the 3′ end of the non-coding strand, and wherein the affinity labeled ds-DNA molecules comprise an affinity labeled strand;
contacting the amplified affinity labeled ds-DNA molecules with an affinity label-binding molecule and selecting amplified affinity labeled ds-DNA molecules which bind the affinity labeled-binding molecule to provide affinity label-binding ds-DNA molecules; and
isolating the non-affinity labeled template strand from the affinity label-binding ds-DNA molecules to provide a first amplified single-stranded DNA template for the selected fully modified RNA aptamers,
wherein a fully modified RNA molecule comprises 2′-fluoro-nucleotides, 2′-methoxy-nucleotides, or a combination of 2′-fluoro-nucleotides and 2′-methoxy-nucleotides, and no un-modified nucleotides.

2. The method of claim 1, wherein the affinity label comprises biotin, and the affinity label binding molecule is a biotin binding molecule.

3. The method of claim 1, further comprising
providing the first amplified single-stranded DNA comprising a template for the selected fully modified RNA aptamers, and preparing a second pool of oligonucleotide complexes that each comprise a ds-DNA molecule tethered to a fully modified RNA molecule, wherein the ds-DNA molecule comprises a coding strand and a complementary non-coding strand which encode the RNA molecule, and
repeating the exposing, selecting, amplifying, contacting and isolating steps to provide a second amplified single-stranded DNA comprising a template for the selected fully modified RNA aptamers.

4. The method of claim 1, wherein the 2′-fluoro-nucleotides and/or 2′-methoxy-nucleotides comprise one or more alkyne-containing nucleotides.

5. The method of claim 1, wherein one or more U, A, G or C nucleotides is alkyne-substituted.

6. The method of claim 5, wherein the fully modified RNA molecule is glycosylated.

7. The method of claim 1, wherein the fully modified RNA molecule is covalently linked to a branched or unbranched oligosaccharide.

8. The method of claim 1, wherein the first pool of oligonucleotide complexes is a selection library made by a method comprising
providing a first single-stranded DNA comprising a random library template flanked by 3′ and 5′ constant primer regions;
annealing the 3′ constant primer region of the single-stranded DNA to a phosphorylated overhanging regeneration primer and adding dNTPs and a first DNA polymerase to provide a bidirectionally extended duplex DNA having a phosphorylated stand and an unphosphorylated strand;
digesting the phosphorylated strand with an exonuclease to provide the unphosphorylated strand, wherein the unphosphorylated strand is a single-stranded hairpin template comprising a 3′ hairpin, the random library template, and the 5′ constant primer region;
extending the hairpin template using a second DNA polymerase and either all 2′-F-NTPs and/or 2′-methoxy-NTPs to provide a modified randomized nucleotide strand complementary to the random library template and the 5′ constant primer region and provide a double stranded hairpin structure; and
displacing the modified randomized nucleotide strand of the double stranded hairpin structure by treatment with a hairpin primer complementary to the 3′ hairpin sequence and extending the hairpin primer with a third DNA polymerase to provide the selection library containing the modified randomized nucleotide strand tethered to a double stranded DNA molecule comprising a coding strand and a complementary non-coding strand which encodes the RNA molecule,
wherein the second DNA polymerase incorporates 2′-fluoro-nucleotides, 2′-methoxy-nucleotides, or a combination of 2′-fluoro-nucleotides and 2′-methoxy-nucleotides.

9. The method of claim 8, wherein extending the hairpin template uses all 2′-F-NTPs, and the second DNA polymerase comprises an engineered Thermococcus sp. 9N-7 DNA polymerase with an A485L mutation.

10. The method of claim 8, wherein extending the hairpin template uses all 2′-methoxy-NTPs, and the second DNA polymerase comprises a Thermococcus kodakarensis KOD1 polymerase with N210D/Y409G/A485L/D614N/E664K mutations.

11. The method of claim 8, wherein the first amplified single-stranded DNA comprising the template for the selected fully modified RNA aptamers is regenerated to form a second single-stranded DNA comprising a second random library template flanked by 3′ and 5′ constant primer regions.

12. A method of making a selection library, comprising
providing a single-stranded DNA comprising a random library template flanked by 3′ and 5′ constant primer regions;
annealing the 3′ constant primer region of the single-stranded DNA to a phosphorylated overhanging regeneration primer and adding dNTPs and a first DNA polymerase to provide a bidirectionally extended duplex DNA having a phosphorylated stand and an unphosphorylated strand;
digesting the phosphorylated strand with an exonuclease to provide the unphosphorylated strand, wherein the unphosphorylated strand is a single-stranded hairpin template comprising a 3′ hairpin, the random library template, and the 5′ constant primer region;
extending the hairpin template using a second DNA polymerase and either all 2′-F-NTPs and/or 2′-methoxy-NTPS to provide a modified randomized nucleotide stand complementary to the random library template and the 5′ constant primer region and provide a double stranded hairpin structure; and
displacing the modified randomized nucleotide strand of the double stranded hairpin structure by treatment with a hairpin primer complementary to the 3′ hairpin sequence and extending the hairpin primer with a third DNA polymerase to provide the selection library containing the modified randomized nucleotide strand tethered to a double stranded DNA molecule comprising a coding strand and a complementary non-coding strand which encodes the RNA molecule, wherein the second DNA polymerase incorporates 2′-fluoro-nucleotides, 2′-methoxy-nucleotides, or a combination of 2′-fluoro-nucleotides and 2′-methoxy-nucleotides.

13. The method of claim 12, wherein the exonuclease comprises a first exonuclease to digest primer and a second exonuclease to digest the phosphorylated strand.

14. A fully modified RNA aptamer that binds to a target molecule selected by the method of claim 1.

15. A fully modified RNA aptamer that binds to a target molecule selected by the method of claim 1, wherein the fully modified RNA aptamer is conjugated to an amplified single-stranded DNA comprising a template for the RNA.

16. A fully modified RNA aptamer that binds to a target molecule selected by the method of claim 1, wherein the fully modified RNA aptamer is not conjugated to an amplified single-stranded DNA comprising a template for the RNA.


SEQUENCE LISTING (12 sequences)

SEQ ID NO: 1 - DNA, 80 bp, synthetic construct
  source 1..80: mol_type = other DNA
cttgtcgtctcctgtgtgcttnnnnnnnnnnnnnnnnnnnnnnnnncccgtacccgttaaaactccacctcataaccgca

SEQ ID NO: 2 - DNA, 60 bp, synthetic construct
  source 1..60: mol_type = other DNA
  modified_base 1: mod_base = OTHER; note = 5' phosphorylated
cccgtacccgaatataaaataaaaatataaaatataaaattgcggttatgaggtggagtt

SEQ ID NO: 3 - DNA, 60 bp, synthetic construct
  source 1..60: mol_type = other DNA
taaaataaaaatataaaatataaaattgcggttatgaggtggagttttaacgggtacggg

SEQ ID NO: 4 - DNA, 20 bp, synthetic construct
  source 1..20: mol_type = other DNA
  modified_base 1: mod_base = OTHER; note = 5' biotinylated
tgcggttatgaggtggagtt

SEQ ID NO: 5 - DNA, 21 bp, synthetic construct
  source 1..21: mol_type = other DNA
cttgtcgtctcctgtgtgctt

SEQ ID NO: 6 - DNA, 80 bp, synthetic construct
  source 1..80: mol_type = other DNA
cttgtcgtctcctgtgtgcttcgttcttatgttctcactcgctagccccgtacccgttaaaactccacctcataaccgca

SEQ ID NO: 7 - DNA, 15 bp, synthetic construct
  source 1..15: mol_type = other DNA
  modified_base 1: mod_base = OTHER; note = 5' biotin-TEG
ttctcactcgctagc

SEQ ID NO: 8 - DNA, 80 bp, synthetic construct
  source 1..80: mol_type = other DNA
cttgtcgtctcctgtgtgctttatccgtaggttgcaccgtgggtctcccgtacccgttaaaactccacctcataaccgca

SEQ ID NO: 9 - DNA, 80 bp, synthetic construct
  source 1..80: mol_type = other DNA
cttgtcgtctcctgtgtgctttgacccacggtgcaacctacggatacccgtacccgttaaaactccacctcataaccgca

SEQ ID NO: 10 - DNA, 80 bp, synthetic construct
  source 1..80: mol_type = other DNA
cttgtcgtctcctgtgtgcttcatagggaacccaggtgattggggccccgtacccgttaaaactccacctcataaccgca

SEQ ID NO: 11 - DNA, 56 bp, synthetic construct
  source 1..56: mol_type = other DNA
cttgtcgtctcctgtgtgctttatccgtaggttgcaccgtgggtctcccgtacccg

SEQ ID NO: 12 - DNA, 15 bp, synthetic construct
  source 1..15: mol_type = other DNA
cgggtacgggagacc